(19) United States
(12) Patent Application Publication
(10) Pub. No.: US 2012/0219942 A1
(43) Pub. Date: Aug. 30, 2012

(54) Methods Employing McrA to Detect 5-Methyl Cytosine

(75) Inventors: John J. Dunn, Bellport, NY (US); Eli Hatchwell, Winchester (GB)

(73) Assignee: Brookhaven Science Associates, LLC, Upton, NY (US)

(21) Appl. No.: 13/323,962

(22) Filed: Dec. 13, 2011

Related U.S. Application Data
(60) Provisional application No. 61/423,453, filed on Dec. 15, 2010.

(51) Int. Cl. C12Q 1/68

(52) U.S. Cl. 435/6.11

(57) ABSTRACT

The invention provides methods for using the rMcrA protein, and derivatives thereof, for direct or semi-direct determination of the methylation status of CpG dinucleotides in methyl-CpG island sequences of interest.

This application claims benefit from U.S. Provisional Application 61/423,453 filed Dec. 15, 2010, the entire contents of which are incorporated herein by reference.

This invention was made with Government support under contract number DE-AC02-98CH10886, awarded by the U.S. Department of Energy and sponsored in part by NIH grant 1U01A1056480-05. The Government has certain rights in the invention.

### BACKGROUND

### Epigenetics—Methylation of CpG Dinucleotides

Epigenetic modification of DNA has emerged as one of the most important links between the environment, life style, and changes in gene expression. Methylation at the C5 position in cytosine residues in CpG dinucleotides to form m⁵CpG (hereinafter represented as m⁵CpG, 5-Me-CpG, 5mCpG, meCpG, or mCpG) or loss of methylation to convert m⁵CpG to CpG are particularly significant epigenetic changes.

Many CpG dinucleotides cluster into 0.5-1.0 kb DNA segments termed CpG islands (CGIs), of which the human genome has ˜30,000, accounting for about 10% of the total DNA. About half of the CGIs are located near promoters and annotated transcription start sites (TSS-CGIs). Methylation of the CGIs of these start sites induces long-term changes in gene expression by directly interfering with transcription factor binding and via gene silencing, wherein the action of mCpG-binding proteins that recruit chromatin-remodeling enzymes form “closed” heterochromatin, thereby preventing transcription. In normal tissues, TSS-CGIs usually are unmethylated but a subset reproducibly becomes aberrantly methylated in cancerous cells.

Many non-TSS CpG clusters in the human genome are associated with repetitive elements that are normally heavily methylated and which are, therefore, transcriptionally silent. The main repeat families, i.e., short interspersed nuclear elements (SINEs), long interspersed nuclear elements (LINEs) and long terminal repeats (LTRs) constitute approximately 27%, 12%, and 7% of CpG dinucleotides, respectively. In contrast to the hypermethylation of TSS-CGIs during tumorigenesis, many of the non-TSS clusters become hypomethylated during tumorigenesis, a change that can activate the expression of repetitive elements, which in turn may foster DNA breakage and genome instability two of the known hallmarks of cancer.

Consequently, there is considerable interest in determining global DNA methylation patterns in normal and diseased tissues.

Presently, detecting methylated- and unmethylated-CpG dinucleotides at the single nucleotide level is labor intensive, and requires considerable expertise and/or costly equipment. The several approaches for determining DNA methylation patterns fall into three major categories: Restriction enzyme-based-, microarray-based-, and bisulfite-based modification, followed by DNA sequencing.

Bisulfite sequencing employs an initial step of bisulfite modification followed by PCR amplification wherein T replaces the nonmethylated cytosines, leaving unchanged the methylated cytosines, which are resistant to modification. After treatment, multi-locus DNA regions of interest typically are amplified using sets of region-specific primers, and the resulting amplicons are analyzed individually using a variety of techniques to identify C→T transitions or unmodified C positions, which correspond, respectively, to unmethylated- and methylated-cytosines in the native DNA. Because of costs, extensive multi-locus PCR analysis is prohibitive for most research and clinical diagnostic laboratories. Further, this approach is problematic when dealing with clinical samples because relatively large quantities of DNA are required for bisulfite treatment when the end point is a multi-locus methylation analysis.

More recently, shotgun sequencing of bisulfite-converted genomic DNA using high-throughput Illumina/Solexa™ sequencing was used to generate massive amounts of epigenomic data; however, this type of method is very expensive, and mapping of the resulting short-sequence-reads to their respective genomic loci is challenging, and again beyond the scope of most clinical diagnostic laboratories.

m⁵CpG Binding Protein McrA

In attempting to moderate these drawbacks, we recently asked whether we could use m⁵CpG binding proteins for sequence-based epigenomic analysis.

Wild-type E. coli K-12 strains possess several restriction systems in addition to the classical EcoK hsdR/M/S host-specificity restriction-modification mechanism. One of these for methylated adenine recognition and restriction (Mrr) has been reported to restrict DNA containing N⁶-methyladenine and also DNA with C⁵-methylcytosine residues (m⁵C) (see Heitman et al. (1987) J. Bacterial. 169:3243-3250; Kelleher, et al. (1991) J. Bacterial. 173:5220-5223; Waite-Rees et al. (1991) J. Bacteriol. 173:5207-5219; and Kretz et al. (1991) J. Bacterial. 173:4707-4716). Neither system restricts DNA methylated by the resident E. coli enzymes encoded by dam, which methylates the A residue in the sequence GATC, or by dcm, which modifies the internal cytosine in CCWGG sequences (W is A or T) at the C⁵ position (see also Raleigh et al. (1986) Proc. Natl. Acad. Sci. USA 83:9070-9074).

DNA containing C⁵-methylcytosine (m⁵C) or 5-hydroxymethyl cytosine (Hm⁵C) is also restricted by the Mcr (for modified cytosine restriction) system which is identical to the previously described Rgl (for restricts glucose-less phage) restriction system that blocks the growth of T-even phages, but only when they contain Hm⁵C, i.e., when their Hm⁵C DNA residues are not glucosylated (Revel (1967) Virology 31:688-701; Fleischman et al (1976) J. Biol. Chem. 251:1561-1570; and Raleigh et al. (1989) Genetics 122:279-296). Later work further subdivided the Mcr system into two genetically distinct regions: McrA (equal to RglA) on an easily excisable but defective lambdoid prophage element e14 located at 25 min on the E. coli K-12 chromosome (Raleigh et al., 1989; Mehta et al (2004) BMC Microbial 4:4) and McrB (or RglB) at map position 99 min in a region that includes the EcoK restriction/modification and Mrr systems (see also Raleigh et al (1988) Nucleic Acids Res. 16:1563-1575). McrA recognizes DNA containing C⁵-methylcytosine or C⁵-hydroxymethylcytosine while McrB also recognizes DNA containing N⁴-methylcytosine. The mcrB locus encodes two polypeptides McrB and C which together function as a nuclease recognizing in cis two half sites 5′-G/A 5mC (N₄₀₋₃₀₀₀) G/A 5mC-3′. Cleavage requires GTP hydrolysis and occurs at a non-fixed distance (˜30 nucleotides) between the two methylated half sites (Stewart et al (1998) Biol. Chem. 379:611-616; Panne et al. (1999(J. Mol. Biol. 290:49-60).

Early studies showed that DNAs methylated by M.HpaII (Cm⁵CGG), M.Eco1831I (Cm⁵CSGG where S is C or G) and M.SssI (m⁵CG) are restricted by the McrA system (see also Kravetz et al. (1993) Gene 129:153-154) and further studies demonstrated that clones expressing the McrA open reading frame conferred both McrA and RglA phenotypes on mcr minus E. coli strains (Raleigh et al (1989). Several indirect studies suggest that McrA is a member of the ββα-Me finger superfamily of nucleases. Its ββα-Me finger also contains an HNH motif common to homing endonucleases as well as many restriction and DNA repair enzymes. The core ββα-Me domain of McrA (residues 159 to 272 of the 277 amino acid long polypeptide) was modeled by Bujnicki and coworkers using a protein sequence threading approach (Bujnicki et al (2000) Mol. Microbiol. 37:1280-1281). This region contains three histidine residues (H-228, 252, and 256) predicted to coordinate a Mg²⁺ ion, as well as four cysteine residues (C-207, 210, 248, and 251) which are thought to form a zinc finger, most likely involved in coordinating Zn²⁺ or some other divalent metal ion to help stabilize the protein's structure and/or help catalyze nuclease activity.

While McrA is predicted to function as a nuclease and induces the DNA damage response (an index of cleavage activity) when a suitable substrate is present (Anton et al. (2004) J. Bacterial 186:5699-5707) McrA-mediated nuclease activity in vitro has never been demonstrated and to date the mechanism for McrA's biological restriction of modified phage or plasmid DNAs is not known. Recently we reported on the cloning, expression, purification and initial characterization of full-length, biologically active rMcrA (Mulligan and Dunn (2008) Protein & Expr. Purif. 62:98-103). However, while all attempts to demonstrate that rMcrA is a nuclease acting on m⁵C-containing DNA were unsuccessful, electrophoretic mobility shift analysis demonstrated that purified rMcrA interacts specifically with DNA fragments containing Cm⁵CGG sequences. This prompted us to design experiments to determine the spectrum of endogenously methylated McrA targets in human DNA. In order to address this question we successfully employed McrA fused to an eight-amino-acid long StrepII tag (rMcrA-S) to affinity-capture methylated restriction fragments from total human DNA. Standard sequencing of these fragments, together with bisulfite genome-sequencing analysis of their original loci in total human DNA revealed more fully the DNA-binding profile of McrA. We also used rMcrA-S in electrophoretic mobility shift assays (EMSAs) with symmetrically methylated, hemimethylated-, and non-methylated-double-stranded DNA probes with a canonical HpaII site and various single base-pair permutations flanking the central m⁵CpG dinucleotide or opposite the m⁵C residue. Together, these data help define the minimal recognition sequence and base-pairing requirements for McrA's interaction with DNA.

### BRIEF DESCRIPTION OF THE DRAWINGS

FIG. 1: CpG Island of the PITX2 gene.

FIG. 2: Affinity purification of the CpG Island of the PITX2 gene.

FIG. 3: Interrogation of the methylation status of HpaII sites in the CpG island of the PITX2 gene.

### EXEMPLIFICATION

Oligonucleotides:

Oligonucleotides were purchased from Integrated DNA Technologies. Individual oligonucleotides with and without m⁵C were dissolved in 10 mM Tris-HCl, 0.01 mM EDTA buffer, pH 7.5, and, as needed, annealed at 36 μM with their complements, in 1× One-Phor-All Buffer (10 mM Tris-Acetate pH 7.5, 10 mM Mg-Acetate, 50 mM K-Acetate) (GE Healthcare) by heating for 2 min at 98° C., then slow cooling to room temperature. With 10%-polyacrylamide gel (PAGE) analysis, we verified their conversion to duplexes, leaving only minimal amounts (<56) of residual single-stranded oligonucleotides.

Tables 2a and b list the oligonucleotides used for this study and give the sequences of their resulting fully base-paired ds-cassettes. C in Tables 2a and 2b indicates a m⁵C residue.

TABLE 2a

| Oligo | |
|---|---|
| Number | 5′-3′ Sequence |
| 1 | GCCTTCAGCGCCGGCGGATCCAGT |
| 2 | ACTGGATCCGCCGGCGCTGAAGGC |
| 3 | GCCTTCAGCGCCGGCGGATCCAGT |
| 4 | ACTGGATCCGCCGGCGCTGAAGGC |
| 5 | GCCTTCAGCGCCGGCGGATCCAGT |
| 6 | ACTGGATCCGCCGGCGCTGAAGGC |
| 7 | GCCTTCAGCGCCGGCGGATCCAGT |
| 8 | ACTGGATCCGCCGGCGCTGAAGGC |
| 9 | CCCTCTGACGGAGGAGGCTCCTGC |
| 10 | GCAGGAGCCTCCTCCGTCAGAGGG |
| 11 | CCCTCTGACGCAGGAGGCTCCTGC |
| 12 | GCAGGAGCCTCCTGCGTCAGAGGG |
| 13 | CCCTCTGACGAAGGAGGCTCCTGC |
| 14 | GCAGGAGCCTCCTTCGTCAGAGGG |
| 15 | CCCTCTGACGTAGGAGGCTCCTGC |
| 16 | GCAGGAGCCTCCTCAGTCAGAGGG |
| 17 | CCCTCTGCCGGAGGAGGATCCTGC |
| 18 | GCAGGATCCTCCTCCGGCAGAGGG |
| 19 | CCCTCTGTCGGAGGAGGCTCCTGC |
| 20 | GCAGGAGCCTCCTCCGACAGAGGG |
| 21 | CCCTCTGGCGGAGGAGGCTCCTGC |
| 22 | GCAGGAGCCTCCTCCGCCAGAGGG |
| 23 | CCCTCTGTCGAAGGAGGCTCCTGC |
| 24 | GCAGGAGCCTCCTTCGACAGAGGG |
| 25 | CCCTCTGGCGAAGGAGGCTCCTGC |
| 26 | GCAGGAGCCTCCTTCGCCAGAGGG |
| 27 | CCCTCTGCCGAAGGAGGCTCCTGC |
| 28 | GCAGGAGCCTCCTTCGGCAGAGGG |
| 29 | ACTGGATCCGCCAGCGCTGAAGGC |
| 30 | ACTGGATCCGCCCGCGCTGAAGGC |
| 31 | ACTGGATCCGCCTGCGCTGAAGGC |
| 32 | ACTGGATCCGCCUGCGCTGAAGGC |
| 33 | ACTGGATCCGCCGGCGCTGAAGGC |

TABLE 2b

| | | rMcrA- |
|---|---|---|
| | | S |
| Cassette | Homo Duplex Sequences | Bound? |
| CG, CCGG, CGG | 5′-GCCTTCAGCGCCGGCGGATCCAGT-3′ | Yes |
| | 3′-CGGAAGTCGCGGCCGCCTAGGTCA-5′ | |
| CCGG | 5′-GCCTTCAGCGCCGGCGGATCCAGT-3′ | Yes |
| | 3′-CGGAAGTCGCGGCCGCCTAGGTCA-5′ | |
| CGG | 5′-GCCTTCAGCGCCGGCGGATCCAGT-3′ | Yes |
| | 3′-CGGAAGTCGCGGCCGCCTAGGTCA-5′ | |
| Unmethylated | 5′-GCCTTCAGCGCCGGCGGATCCAGT-3′ | No |
| | 3′-CGGAAGTCGCGGCCGCCTAGGTCA-5′ | |
| CGG and ACGG | 5′-CCCTCTGACGGAGGAGGCTCCTGC-3′ | Yes |
| | 3′-GGGAGACTGCCTCCTCCGAGGACG-5′ | |
| CGC | 5′-CCCTCTGACGCAGGAGGCTCCTGC-3′ | No |
| | 3′-GGGAGACTGCGTCCTGGCAGGACG-5′ | |
| CGA and ACGA | 5′-CCCTCTGACGAAGGAGGCTCCTGC-3′ | Yes |
| | 3′-GGGAGACTGCTTCCTCCGAGGACG-5′ | |
| CGT | 5′-CCCTCTGACGTAGGAGGCTCCTGC-3′ | No |
| | 3′-GGGAGACTGCATCCTCCGAGGACG-5′ | |
| CCGG | 5′-CCCTCTGCCGGAGGAGGATCCTGC-3′ | Yes |
| | 3′-GGGAGACGGCCTCCTCCTAGGTGC-5′ | |
| TCGG | 5′-CCCTCTGTCGGAGGAGGCTCCTGC-3′ | Yes |
| | 3′-GGGAGACAGCCTCCTCCGAGGACG-5′ | |
| GCGG | 5′-CCCTCTGGCGGAGGAGGCTCCTGC-3′ | Yes |
| | 3′-GGGAGACCGCCTCCTCCGAGGACG-5′ | |
| TCGA | 5′-CCCTCTGTCGAAGGAGGCTCCTGC-3′ | Yes |
| | 3′-GGGAGACAGCTTCCTCCGAGGACG-5′ | |
| GCGA | 5′-CCCTCTGGCGAAGGAGGCTCCTGC-3′ | Yes |
| | 3′-GGGAGACCGCTTCCTCCGAGGACG-5′ | |
| CCGA | 5′-CCCTCTGCCGAAGGAGGCTCCTGC-3′ | Yes |
| | 3′-GGGAGACGGCTTCCTCCGAGGACG-5′ | |

Oligonucleotides 1-6 (Table 2a) were synthesized with the m⁵C residue at various positions, while the remaining oligonucleotides were methylated post-synthesis, when needed, using the ds-specific CpG methylase (M.SssI) after annealing to form duplexes, as described above. A typical reaction (20 μl) included 180 pMol duplex DNA in 1×SssI buffer (10 mM Tris-HCl, 50 mM NaCl, 10 mM MgCl₂, and 1 mM dithiothreitol) supplemented with 160 μM S-adenosylmethionine (SAM). Reactions were started by adding 8 units of M.SssI enzyme and incubating the mix overnight at 37° C., then spiking with additional SAM for a final concentration of 320 μM, and finally incubating the samples at 37° C. for another 2 hrs. The M.SssI was inactivated by heating at 65° C. for 20 min. To check that methylation was complete, we included as a control a duplex containing a single HpaII (CCGG) site, and no other CpG dinucleotides. We then checked that the DNA was protected against digestion by HpaII (methyl sensitive), but was susceptible to digestion by the methyl-insensitive isoschizomer MspI. For each assay, 2 μl of methylation reaction was added to 10 mM Bis-Tris-Propane-HCl, 10 mM MgCl₂, 1 mM dithiothreitol supplemented with 100 μg/ml BSA, followed by 10 units of either HpaII or MspI enzyme. Reactions were incubated at 37° C. for 2 hrs before being analyzed by agarose-gel electrophoresis. Gels were stained with ethidium bromide and the DNA bands visualized under UV light.

Table 3 shows the oligonucleotide cassettes used to determine the binding of rMcrA-S to double-stranded (ds)-cassettes with a mismatched base opposite the m⁵C residue.

TABLE 3

| | | rMcrA- |
|---|---|---|
| | | S |
| Cassette | Hetero Duplex Sequences | Bound? |
| m⁵C control | 5′-GCCTTCAGCGCCGGCGGATCCAGT-3′ | Yes |
| | 3′-CGGAAGTCGCGGCCGCCTAGGTCA-5′ | |
| C/G | 5′-GCCTTCAGCGCCGGCGGATCCAGT-3′ | Yes |
| | 3′-CGGAAGTCGCGGCCGCCTAGGTCA-5′ | |
| C/A | 5′-GCCTTCAGCGCCGGCGGATCCAGT-3′ | No |
| | 3′-CGGAAGTCGCGACCGCCTAGGTCA-5′ | |
| C/C | 5′-GCCTTCAGCGCCGGCGGATCCAGT-3′ | No |
| | 3′-CGGAAGTCGCGCCCGCCTAGGTCT-5′ | |
| C/T | 5′-GCCTTCAGCGCCGGCGGATCCAGT-3′ | No |
| | 3′-CGGAAGTCGCCTCCGCCTAGGTCA-5′ | |
| C/U | 5′-GCCTTCAGCGCCGGCGGATCCAGT-3′ | No |
| | 3′-CGGAAGTCGCGUCCGCCTAGGTCA-5′ | |
| C/I | 5′-GCCTTCAGCGCCGGCGGATCCAGT-3′ | Yes |
| | 3′-CGGAAGTCGCGICCGCCTAGGTCA-5′ | |
| Unmethylated | 5′-GCCTTCAGCGCCGGCGGATCCAGT-3′ | No |
| control | 3′-CGGAAGTCGCGGCCGCCTAGGTCA-5′ | |

Strains and Enzymes.

E. coli. Top10 (Invitrogen) was used for DNA manipulation and plasmid preparations. Strain BL21-AI was obtained from Invitrogen. All enzymes, unless otherwise stated, were purchased from New England Biolabs.

rMcrA-S.

The 290-amino-acid McrA coding sequence, with either an 8 amino-acid N terminal or a C terminal StrepII tag (Schmidt, et al., (Nat. Protoc. 2:1528-1535) (underlined) (MASWSHPQFEKGA-start of McrA or end of McrA-SAWSHPQFEK, respectively) were constructed by PCR amplification from a wild-type, untagged mcrA clone. We used primers that appended the StrepII tag and unique restriction sites to aid in cloning on the ends of the PCR product. After restriction-enzyme digestion and gel-purification, the amplicons were cloned into pET28. We verified the accuracy of the clones by DNA sequencing and then moved them into the expression host BL21(DE3)/pRIL; the recombinant proteins were expressed following autoinduction at 20° C. as previously described (Mulligan et al., 2008). The departure here was that after SP-Sepharose Fast Flow (Pharmacia Biotech) chromatography, we further purified the recombinant proteins by binding them to Strep Tactine SpinPrep™ filters (Novagen), followed by elution with LEW buffer (50 mM NaPO₄, pH 8.0; 300 mM NaCl) containing 10 mM biotin. The purified proteins were stored at 4° C. Preliminary studies indicated that the N-terminal tagged protein (S-rMcrA) was markedly less efficient in binding HpaII-methylated DNA fragments to streptavidin-coated magnetic beads (Nanolink) than was its C-terminal analogue (rMcrA-S); therefore, the latter was utilized in all remaining experiments.

rMcrA-S Affinity Enrichment of DNA Fragments Containing m⁵CpG.

Human genomic A549 DNA (lung carcinoma) was digested exhaustively with MseI having recognition sites (TTAA) that rarely occur in GC-rich regions, so that most regions with a high density of m⁵CpG dinucleotides were left intact. The digested DNA was phenol-extracted, precipitated with ethanol, and then dissolved in 10 mM Tris-HCl, 0.01 mM EDTA (TEsl), pH 8.0. Approximately 750 ng of fragmented DNA was incubated for 20 min at room temperature (RT) with ˜7 nmol of rMcrA-S in 200 μl LEW buffer supplemented with 100 μg/ml BSA and 250 ng sonicated E. coli. ER2925 (dam⁻, dcm⁻) DNA as the carrier. Then we added a 25 μl bed-volume of Nanolink magnetic strepavidin beads, prewashed twice in 1×LEW+100 μg/ml BSA, and incubated the materials at RT for 1 hour with gentle mixing to capture the rMcrA-S/A549 DNA complexes. The unbound fraction was removed and the beads were washed 3× with 100 μl 1×HEPES+250 mM NaCl; 3× with 1×HEPES+700 mM NaCl; and, 2× with 50 μl 1×HEPES+250 mM NaCl. The beads with the bound DNA fragments were then washed in 50 μl 1× Quick Ligase Buffer (NEB) (66 mM Tris-HCl, 10 mM MgCl₂, 1 mM dithiothreitol, 1 mM ATP and 7.5% (W/V) polyethylene glycol (PEG 6000) and resuspended in 50 μl 1× Quick Ligase Buffer.

Ligation-mediated PCR (LM-PCR).

A MseI-compatible adaptor DNA cassette was formed by annealing two oligonucleotides: MseI Top: 5′-AGCAACTGTGCTATCCGAGGGAT-3′, and MseI Bottom: 5′-TAATCCCTCGGA-3′ and then ligating the product to the MseI-compatible ends of the DNA captured on the strepavidin beads by rMcrA-S. We added 100 pmol of adaptor and 3000 units of T4 DNA ligase to the resuspended magnetic beads in 50 μl 1× Quick Ligation Buffer. The reaction was incubated at 16° C. overnight.

The beads then were washed and equilibrated with 100 μl 1× Thermo Pol Buffer (NEB) (20 mM Tris-HCl pH 8.8, 10 mM KCl, 10 mM (NH₄)₂SO₄, 2 mM MgSO₄, 0.1% Triton X-100). PCR amplification was done in 50 μl NEB Thermo Pol buffer with Taq polymerase using a single unphosphorylated primer: 5′-AGCAACTGTGCTATCCGAGGGAT-3′. The reactions were started by incubating the material at 72° C. for 10 min to fill-in the single stranded regions of the appended cassettes, and then were cycled 14 times at 94° C. for 20 sec, 68° C. for 30 sec, and 72° C. for 2 min 30 sec. The amplified products were purified using Qiagen PCR Purification Kit columns (Qiagen), and resuspended in 20 μl of distilled H₂0 with 50 mM HEPES (pH 7.5). Linkered fragments were cloned using a pSMART GC kit (Lucigen). Individual recombinant clones were sequenced using standard ABI dideoxy sequencing.

Bisulfite Sequencing.

Genomic A459 DNA was bisulfite-converted according to the manufacturer's instructions (Qiagen), followed by whole-genome amplification (Qiagen), omitting the initial denaturing steps since the DNA already was single-stranded after bisulfite conversion. Primers were designed (http://www.urogene.org/methprimer/indexl.html) for two regions of the bisulfite-modified genomic DNA. Region C1 was amplified using the forward- and reverse-primers, C1F3: 5′-TGGGGTGTTTTTTTTGTATT-3′ and C1R1: 5′-AAAATCCCACCCTAAACC-3′ respectively and region C2 with the forward and reverse primers C2F3: 5′-TGGGTTTTGTATAGGTTAAA-3′ and C2R1: 5′-AACAACCAAAAAATTTTCAC-3′, respectively. PCR was done following the manufacturer's conditions, using NEB Thermo Pol buffer and Taq polymerase supplemented with 5% DMSO (final concentration) and amplified as follows: 94° C. for 2 min, 2 cycles of (94° C. for 30 sec, 55° C. for 30 sec, 72° C. for 2 min) 2 cycles of (94° C. for 20 sec, 54° C. 30 sec, 72° C. for 2 min) 2 cycles of (94° C. for 20 sec, 53° C. for 30 sec, 72° C. for 2 min) 2 cycles of (94° C. for 20 sec, 52° C. for 30 sec, 72° C. for 2 min) and 30 cycles of (94° C. for 20 sec, 51° C. for 30 sec, 72° C. for 2 min). PCR products were gel purified using PCR purification columns (Qiagen), ligated into pCR4TOPO vector (Invitrogen) via manufacturer's standard protocol, electroporated into E. coli Top 10 cells, and plated on 2×YT supplemented with 50 μg kanamycin. We picked individual colonies and grew them in 2×YT media with 50 μg kanamycin. Plasmids were isolated using alkaline lysis followed by column purification (Fermentas), and sequenced with primers flanking the cloning site. Sequencher software (Gene Codes) was employed to edit the sequences, and CLUSTLW to analyze them.

EMSA binding assays. Reactions (10 μl) typically contained a mixture of 10-25 pMol of various test DNAs, and 250 ng sonicated E. coli. ER2925DNA as a non-methylated, non-specific competitor in 50 mM Tris-HCl, 100 mM NaCl, 1 mM dithiothreitol, 10 mM MgCl₂ (NEBuffer #3), supplemented, with 100 μg/ml BSA. We started the reactions adding 25-175 pMol McrA-S; after 45 min at RT, the entire sample was loaded on 10% acrylamide Tris-Acetate/EDTA gel (1×TAE: 40 mM Tris-Acetate, 1 mM disodium EDTA) which was electrophoresed at RT. Thereafter, the gels were stained with ethidium bromide (0.5 μg/ml) at RT and visualized with UV light. Then, we stained the gels with Coomassie Blue to detect rMcrA-S.

### Findings

The human genome contains about 2.3 million HpaII sites of which roughly 12%, are located in regions with a density of m⁵CpG dinucleotides, regions referred to as CpG islands (CGIs) (see Table 1—Supplementary Material of Mulligan, et al. 2010). The growing interest in determining global DNA methylation patterns in normal- and abnormal-tissues has been addressed by a number of methods. Widely used methods for such studies include immunoprecipitation; methylation specific PCR; m⁵CpG affinity-capture using m⁵CpG-binding proteins and related methods, restriction enzyme-based methods, microarray-based methods or various combinations of these methods and bisulfite-based modification followed by single or multi-locus DNA sequencing or more recently by high-throughput, massively parallel sequencing.

Our initial McrA studies used phage T7 DNA methylated in vitro only at HpaII sites, and therefore we could not ascertain whether McrA can also bind to, and perhaps be used for affinity purification of DNA fragments with related sequences containing m⁵CpG's or, alternatively, if McrA would preferentially enrich a subset of eukaryotic CGIs containing several methylated HpaII sites. A standard technique for converting McrA into such an affinity reagent would be to fuse a (His)₆ tag to either end of the protein and then use the tagged protein to generate an affinity matrix for binding DNA fragments containing m⁵CpG's. However, rMcrA intrinsically binds to Ni-charged NTA supports, presumably because of interaction with three suitably positioned histidines in its ββα-Me domain. Our initial experiments indicated inefficient binding of rMcrA/DNA complexes to Ni-charged NTA magnetic beads, presumably due to steric hindrance. Therefore, we added an eight-amino-acid long StrepII tag (WSHPQFEK) (Schmidt et al., 2007) to one end of the protein to foster the affinity capture of methylated DNA fragments independent of the Ni binding site. In our hands, placing this tag at the C-terminus does not seem to adversely affect the protein's capture of DNA fragments with methylated-HpaII sequences, whereas its N-terminal tagged counterpart is much less effective in binding such sequences.

To further resolve McrA's binding specificity, we initially used rMcrA-S to enrich for methylated sequences from a MseI digest of genomic human DNA because it would contain m⁵CpG dinucleotides in all possible contexts, and further, MseI leaves CGIs mostly intact. In addition, this way we could ascertain whether rMcrA-S preferentially captures CGIs with methylated HpaII sites. Accordingly, we washed bound fragments obtained by rMcrA-S affinity purification with high-ionic-strength buffer, and then LM-PCR-amplified, cloned, and sequenced a subset of the clones to determine if they all contained HpaII sites, and if any originated from chromosome regions defined as being a CGI. We found that most clones from a library of non-size-selected amplicons were not from CGIs, but from regions containing repetitive sequences; i.e., regions known to contain m⁵CpGs. Some minor enrichment for CGI fragments was noted when the amplified DNA was gel-purified and fragments between 0.5 and 2 Kb were used to prepare the library (viz., the size range expected for CGIs in the digest). While many clones in both libraries contained one or more HpaII sites, some had no HpaII sites although they invariably contained several CpG dinucleotides. Overall, we found an approximately 2 to 3-fold increase in CpG dinucleotides in these libraries over the starting DNA.

After we located all affinity captured sequences in the human genome using the UCSC Genome Browser (March, 2006 assembly, http://genome.ucsc.edu/), we chose two unique regions to study further: “C1,” within Ch. 8q24:3, containing 2 HpaII sites, one CGG sequence, and five other CpG dinucleotides; and, “C2,” within Ch. 18q11.2, lacking HpaII sites but having a single CGG sequence plus three other CpGs within the MseI fragment. We next determined the methylation status of these sites in vivo via sodium-bisulfite sequencing. Briefly, total genomic A549 DNA was bisulfite-modified, amplified by a whole-genome amplification assay, and then the C1 and C2 regions were amplified using bisulfite-specific primers C1F3, C1R1, and C2F3, C2R1, respectively. Standard sequencing methods allowed us to deduce the genomic methylation pattern in 12 clones from each region.

Aligning these sequences showed that 63-100% of all CpG sites in C1 were methylated, and all C1 clones had at least one methylated HpaII sequence or m⁵CGG sequence (hereafter referred to as a three-fourth HpaII site). For the C2 clones, from a region lacking HpaII sites, we found that all 12 clones were methylated at the three-fourth HpaII site. Ten of them exhibited 100% methylation across all four CpG dinucleotides. These data led us to investigate whether the three-fourth HpaII site is a minimal binding site for rMcrA-S.

To further define a consensus rMcrA-S DNA recognition site we turned to EMSA using 24 bp synthetic oligonucleotide cassettes containing one of the following: (A) Three m⁵CpGs including one in an HpaII site (Cm⁵CGG); (B) one with a single m⁵C in a ‘three-fourth. HpaII site’ (m⁵CGG); and, (C) one containing a single m⁵C in an HpaII site. As a control, we used a ds-cassette with no m⁵Cs (Table 2b). At the protein/DNA ratios we used, rMcrA-S has high affinity for a ds-cassette containing a single symmetrically methylated HpaII site; however, at higher ratios of protein to DNA, binding to the cassette with a single symmetrically methylated three-fourth HpaII site becomes evident, but no binding to the unmethylated control cassette was observed. In other experiments we found that binding is independent of Mg⁺⁺ ions in the buffer.

Next we used complementary synthetic oligonucleotides, annealed and M.SssI methylated in vitro, as described above to further define rMcrA-S binding preference. As Table 2b illustrates, these oligonucleotides all contain a single CpG dinucleotide either preceded by D (A, G or T) or followed by H (A, C or T). The binding is summarized in Table 2b.

rMcrA-S preferentially shifts cassettes when a purine R follows the m⁵CpG dinucleotide rather than a pyrimidine (Y). These findings suggest why previous in vitro studies revealed that McrA did not bind T7 DNA with methylated HhaI sites (Gm⁵CGC) (Mulligan et al., 2008).

We also investigated the importance of cytosine preceding m⁵CGR. Double-stranded cassettes were designed with a single NCGR site, M.SssI methylated in vitro, and then analyzed for rMcrA-S binding by EMSA. rMcrA-S bound all eight cassettes but seemingly had a somewhat higher affinity for duplexes with Ym⁵CGR while shifting duplex cassettes with an Rm⁵CGR sequence. These results are consistent with our initial findings that rMcrA-S can affinity-purify genomic MseI fragments lacking a methylated HpaII site but containing Am⁵CGG, a three-fourth HpaII site.

### Hemi-Methylated DNA

During mammalian DNA replication, CpG dinucleotides in the daughter strand initially are unmethylated until later methylated by the maintenance methyltransferase, DNMT1 (Bolden et al. (1986) Prog. Nucleic Acid Res. Mol. Biol. 33:231-250). Therefore, we were interested to learn whether rMcrA-S interacts with a hemimethylated Cm⁵CGG sequence. For these studies, we annealed an oligonucleotide with and without a single m⁵C added during synthesis with its methylated- or non-methylated-complement (Table 3). We found that rMcrA-S gel shifts only the ds-cassettes with a fully methylated or hemimethylated HpaII site and not the unmethylated cassette.

We next tested the ability of rMcrA-S to gel-shift ds-cassettes wherein an A, C, T, U, or I residue is placed opposite the m⁵C. Interestingly, EMSA shifts were observed only when a G or I is opposite the m⁵C; no shifted complexes are seen with the others.

From these experiments, we conclude that rMcrA-S can bind ds-DNA fragments with a single symmetrically methylated Cm⁵CGG sequence, or sites where Y precedes, or R follows the methylated central CpG dinucleotide. Electrophoresis of rMcrA-S/DNA complexes on a polyacrylamide gel typically generated two shifted bands. These may represent mono-dimer equilibria of the complexes that form during EMSA but additional studies will be needed to adequately address this. Interestingly, our initial studies indicate that rMcrA-S does not demonstrate high affinity to human genomic CGIs with methylated HpaII sites. Further work is needed with CGI-specific- and other types of microarrays to determine if rMcrA-S can be used for high-resolution DNA methylation analysis of tiled CGIs and to verify if McrA is indeed a nuclease in vivo. To date all tests for double-stranded DNA cleavage by McrA have been negative as have assays for nicking activity on HpeII methylated supercoiled plasmids and for m⁵C DNA glycosylase activity. In addition, since we see no evidence for cleavage on long T7 DNA fragments with numerous symmetrically methylated HpaII sites, we do not believe that McrA is similar to typeIIS restriction endonucleases that cleave outside or between their recognition sites, as is the case for McrBC.

Presumably, McrA acting simply as a DNA binding protein could interfere or “biologically restrict” the maintenance or growth of m⁵C or Hm⁵C modified plasmids and phages by interfering with DNA replication (Anton, et al., 2004) since it should bind in vivo to symmetrically methylated as well as hemimethylated sites. Alternatively binding might not be tightly correlated with cleavage specificity. McrA binding to m⁵C or Hm⁵C modified sites might elicit post-translational modification of McrA, or alternatively the synthesis or interaction with some other E. coli protein(s) needed to catalyze cleavage in vivo. The recombinant McrA-S protein described here should be useful in attempts to identify such putative host protein(s) with which it might interact in vivo to a form noncovalent, catalytically active complex by co-chromatography on a Strep-Tactin® affinity matrix.

### Binding to Hemi-Methylated DNA to Interrogate Methylation Status of CpG Islands

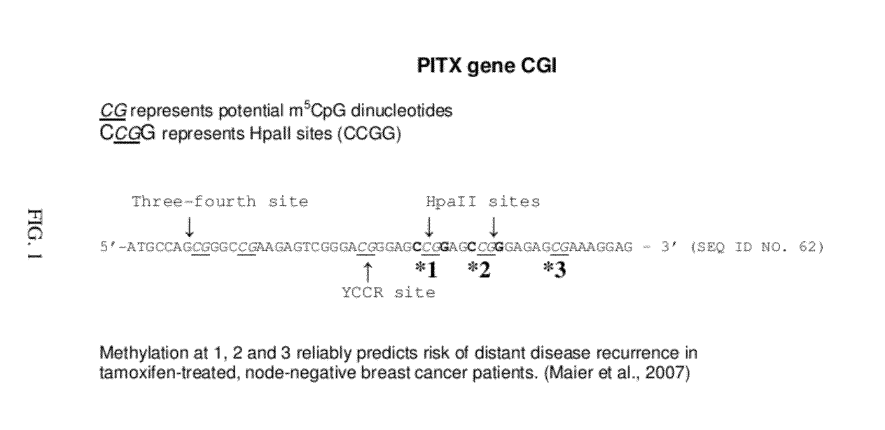

As we envision here (see FIGS. 1-3), region-specific capture oligonucleotides with and without mismatches opposite the diagnostic m⁵CpG sites would be used as probes. Several different probes would be used to interrogate the methylation status of individual CpG dinucleotides covered by each probe set. Detection of bound rMcrA-S would utilize any number of very sensitive ELISA techniques. Alternatively the capture oligos and the denatured test DNA could be annealed in solution followed by addition of rMcrA-S. Samples could then be analyzed by gel electrophoresis to separate the rMcrA-S/DNA complexes from the capture oligo. In this format the capture oligos could be biotinylated or fluorescene-labeled to allow sensitive detection of the gel shifted complexes.

### PITX2 Illustration

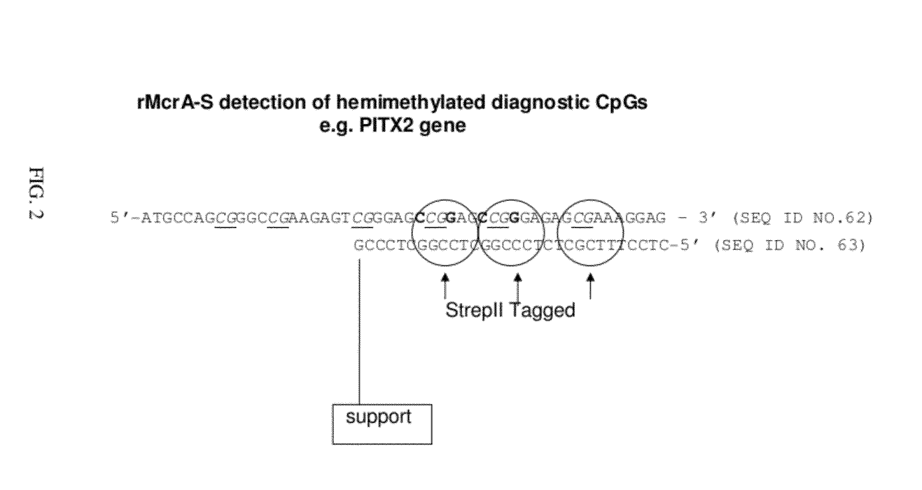

As an example of the potential use of this method, we used for illustration the CGI of PITX2, a homeodomain transcription-factor gene implicated in the progression of breast cancer (FIGS. 1, 2, & 3). The CGI has two HpaII sites (1 & 2) plus a GCGA site (3) that, when methylated, are indicative of a poor patient prognosis (Maier, et al. (2007) Eur. J. Cancer 43:1679-1686).

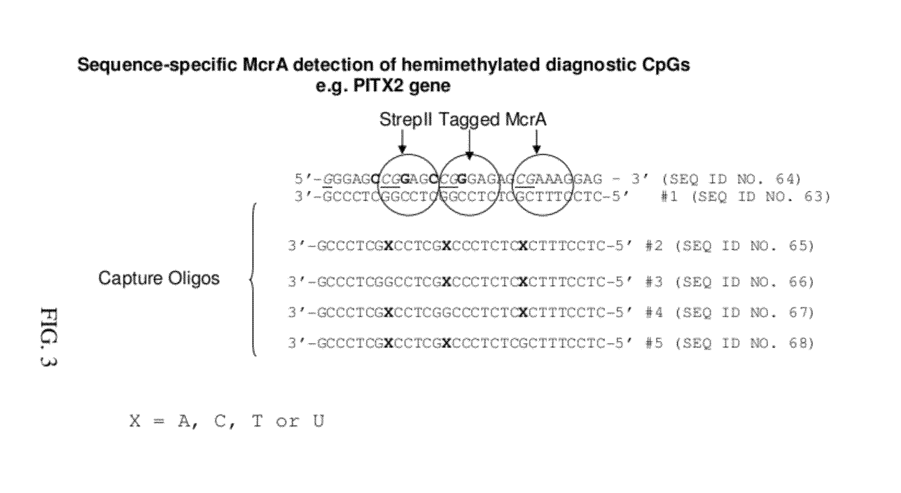

Step 1 would be to fragment the DNA mechanically or with restriction enzyme(s) that cut outside the PITX2 CGI, and then (Step 2—FIG. 2) enrich for fragments with m⁵CpG by affinity-purification using an MBD2 recombinant protein construct. The captured DNA fragments would be denatured and annealed to a tethered set of designed capture oligos (Step 3—FIG. 3). After washing to remove unhybridized DNAs and digestion with a single-strand specific nuclease (e.g. S1) to remove single-stranded regions flanking the captured DNA, the diagnostic m⁵C residues in the captured PITX2 fragments are detected by adding a solution of rMcrA-S in buffer (Step 4). After ˜60 minutes incubation at room temperature, excess rMcrA-S is washed away, and the bound rMcrA-S detected using any one of several ELISA methods (Step 5): McrA antibody, fluorescently labeled streptavidin, streptavidin complexed with biotinylated horseradish peroxidase, Strep-Tactin/enzyme conjugates, or monoclonal antibody to the S tag (StrepMAB-Classic).

In the example shown above (FIG. 3), DNA captured by oligo #1 is expected to bind rMcrA-S at sites 1 and 2 and possibly 3 if they are methylated in the starting DNA sample. No binding should be seen if the PITX2 CGI is unmethylated at all three sites. Likewise, there should be no binding if either methylated- or unmethylated-genomic DNA is captured by oligo #2 since mismatches are opposite all three potential m⁵C residues. This oligo serves as a negative control. The methylation status of each individual site can be differentiated using capture oligos #3, #4, and #5. If all three sites are methylated, then oligos #3, #4, and #5 should give a positive signal. If site 1 only is methylated, then we would see a positive signal only when using oligos #1 and #3 for capture. Similarly, if only site 2 is methylated, then we should obtain a positive signal only when using oligos #1 and #4 for capture: when only site #3 is methylated then a positive signal should be apparent only when using oligos #1 and #5.

An rMcrA-S-based m⁵C detection approach has several potential advantages over current bisulfite-based methods, such as methylation-specific PCR (MSP), or the more labor-intensive cloning and sequencing of bisulfite-modified DNA regions. Importantly, it would eliminate all the tedious bisulfite-modification-steps, as well as concerns about the potential degradation of DNA during the conversion steps, and spurious results from, the incomplete conversion of cytosine to uracil. Finally, after the bisulfite conversion of unmethylated cytosines, usually the PCR products lack C (sense) or G (antisense strand) nucleotides, so that these amplicons have a significantly lower GC content than the initial genomic DNA, thereby confounding the design of proper amplification primers.

In the method, Step 1, fragmenting the DNA sample, the DNA is fragmented to a convenient size range. The fragmentation may be carried out by endonuclease digestion, by restriction endonuclease treatment or by mechanical methods such as sonication, nebulization and/or hydroshear.

Use of specific restriction endonucleases may take the form of selection of restriction endonucleases which are likely to leave most CpG island segments of the DNA intact. Examples include restriction enzymes that recognize and cleave at AT-rich sequences, including MseI, SSpI, Tsp509I, or AseI. Restriction enzyme fragmentation may produce either blunt ended fragments or fragments having sticky ends. A preferable restriction enzyme is MseI, which recognizes and cleaves the sequence TTAA, leaving a TA sticky end for later ligation of an amplification cassette. Optionally the digestion with restriction enzyme may be repeated to ensure full digestion.

Fragmentation of the DNA to a convenient size may be accomplished through mechanical means such as hydroshear, nebulization and/or sonication. DNA fragmented by these mechanical means may require repair of the ends of the DNA to provide blunt ended double stranded DNA fragments. In such circumstances it is preferable to repair the ends so that ligation with a blunt ended amplification cassette is facilitated.

A convenient size range for the double stranded fragments may be from about 100 base pairs to about 2000 base pairs, preferably from about 500 to about 1000 base pairs.

Optionally, fragmented DNA may be separated into two or more portions on the basis of richness or density in Me-CpG regions (i.e., the number of Me-CpG dinucleotides per fragment of a particular size) using Me-CpG binding ligands. Some binding ligands have sufficient avidity to adequately bind, for separation purposes, DNA fragments having as few as one Me-CpG dinucleotide in about sixty base pairs. Solid supports bearing the MBD of MeCP2 can be used to fractionate the DNA fragments into sub-groups by stepwise elution with buffers of increasing salt concentrations (Cross et al, 1994). The Me-CpG DNA fragments are then further processed to enrich for theme-CpG sequences of interest by annealing to a tethered set of appropriately designed capture oligos.

The capture oligos are designed using known or suspected CGI sequences that are or are suspected as being related to a particular disease status, disease progression, treatment success prediction and other prognosis information and etc.

Those skilled in the art will realize that the methods described herein may be applied to any CGI of interest provided the sequence is known. Although only a single CGI (from the PITX2 gene) is specifically illustrated herein, because the method relies upon annealing of complementary (fully and partially complementary) DNA strands the method is universally applicable to any DNA sequence of interest wherein the DNA has CpG dinucleotides. The method herein may be used for diagnostic and prognostic purposes in patients.

1. A method for determining methylation status CpG dinucleotides in CpG island sequences (CGIs) of interest comprising:
a) providing a DNA sample;
b) fragmenting the DNA to form a plurality of double stranded fragments having a convenient size range;
c) enriching for methyl-CpG island sequences of interest from the plurality of fragments;
d) separately annealing the enriched sequences of c) to a series of capture oligonucleotides, each capture oligonucleotide having either full complementarity, partial mis-match, or full mis-match to the m⁵C residue of the CGI to form duplexes; and
e) incubating the annealed duplexes of d) above with recombinant McrA (rMcrA), or derivative thereof, under conditions for binding of rMcrA to symmetrically methylated CpG and hemi-methylated CpG sequences; and
f) quantifying the binding of said rMcrA to each duplex thereby determining the methylation status of CpG dinucleotides in the CpG island sequences (CGIs) of interest.

2. The method according to claim 1 in which the DNA is fragmented through the use of endonuclease digestion, restriction enzyme digestion or mechanical methods.

3. The method according to claim 2 wherein the mechanical methods are selected from the group consisting of hydroshear, sonication and nebulization.

4. The method according to claim 2 in which the DNA is fragmented by restriction enzyme digestion.

5. The method according to claim 4 in which the restriction enzyme digestion is repeated to ensure complete digestion.

6. The method according to claim 1 in which the methyl-CpG island sequences of interest are enriched using the MBD2/MBD3L1 complex.

7. The method according to claim 1 in which the methyl-CpG island sequences of interest are enriched using a bound complementary capture oligonucleotide.

8. The method according to claim 1 in which the convenient size range is from about 100 to about 2000 base pairs.

9. The method according to claim 8 in which the convenient size range is from about 500 to about 1000 base pairs.

10. The method according to claim 1 in which the rMcrA, or derivative thereof, is a fusion protein of rMcrA and an affinity tag.

11. The method according to claim 10 wherein the affinity tag is fused in-frame to the amino terminus of rMcrA.

12. The method according to claim 11 wherein the affinity tag is fused in-frame to the carboxyl terminus of rMcrA.

13. The method according to claim 11 or 12 wherein the affinity tag is selected from the group consisting of GST, S Tag, T7 tag, Strep tag, FLAG, chitin binding domain, and calmodulin binding protein.

14. A method for direct determination of methylation status of CpG dinucleotides in CpG island sequences (CGIs) of interest comprising separately hybridizing said CGIs with complementary and partially complementary oligonucleotide probes to form a series of duplexes such that individual methyl-CpG sequences in the duplexes so formed are either matched or mismatched in the duplexes and differentially binding the duplexes with a methyl-CpG binding protein that recognizes hemi-methylated CpG sequences only if the methyl-C of the methyl-CpG sequence is opposite a matching G or I residue.

15. The method according to claim 14 wherein the methyl-CpG binding protein is McrA or derivative thereof.

16. The method according to claim 14 wherein the methyl-CpG binding protein is rMcrA or derivative thereof.


SEQUENCE LISTING (68 sequences)

SEQ ID NO: 1 - DNA, 24 bp, Artificial Sequence
  Oligonucleotide with 5-MeC at 9, 12 and 15
gccttcagcgccggcggatccagt

SEQ ID NO: 2 - DNA, 24 bp, Artificial Sequence
  Oligonucleotide with 5-MeC at 9, 12 and 15
actggatccgccggcgctgaaggc

SEQ ID NO: 3 - DNA, 24 bp, Artificial Sequence
  Oligonucleotide with 5-MeC at 12
gccttcagcgccggcggatccagt

SEQ ID NO: 4 - DNA, 24 bp, Artificial Sequence
  Oligonucleotide with 5-MeC at 12
actggatccgccggcgctgaaggc

SEQ ID NO: 5 - DNA, 24 bp, Artificial Sequence
  Oligonucleotide with 5-MeC at 15
gccttcagcgccggcggatccagt

SEQ ID NO: 6 - DNA, 24 bp, Artificial Sequence
  Oligonucleotide with 5-MeC at 9
actggatccgccggcgctgaaggc

SEQ ID NO: 7 - DNA, 24 bp, Artificial Sequence
  Oligonucleotide
gccttcagcgccggcggatccagt

SEQ ID NO: 8 - DNA, 24 bp, Artificial Sequence
  Oligonucleotide
actggatccgccggcgctgaaggc

SEQ ID NO: 9 - DNA, 24 bp, Artificial Sequence
  Oligonucleotide
ccctctgacggaggaggctcctgc

SEQ ID NO: 10 - DNA, 24 bp, Artificial Sequence
  Oligonucleotide
gcaggagcctcctccgtcagaggg

SEQ ID NO: 11 - DNA, 24 bp, Artificial Sequence
  Oligonucleotide
ccctctgacgcaggaggctcctgc

SEQ ID NO: 12 - DNA, 24 bp, Artificial Sequence
  Oligonucleotide
gcaggagcctcctgcgtcagaggg

SEQ ID NO: 13 - DNA, 24 bp, Artificial Sequence
  Oligonucleotide
ccctctgacgaaggaggctcctgc

SEQ ID NO: 14 - DNA, 24 bp, Artificial Sequence
  Oligonucleotide
gcaggagcctccttcgtcagaggg

SEQ ID NO: 15 - DNA, 24 bp, Artificial Sequence
  Oligonucleotide
ccctctgacgtaggaggctcctgc

SEQ ID NO: 16 - DNA, 24 bp, Artificial Sequence
  Oligonucleotide
gcaggagcctcctacgtcagaggg

SEQ ID NO: 17 - DNA, 24 bp, Artificial Sequence
  Oligonucleotide
ccctctgccggaggaggatcctgc

SEQ ID NO: 18 - DNA, 24 bp, Artificial Sequence
  Oligonucleotide
gcaggatcctcctccggcagaggg

SEQ ID NO: 19 - DNA, 24 bp, Artificial Sequence
  Oligonucleotide
ccctctgtcggaggaggctcctgc

SEQ ID NO: 20 - DNA, 24 bp, Artificial Sequence
  Oligonucleotide
gcaggagcctcctccgacagaggg

SEQ ID NO: 21 - DNA, 24 bp, Artificial Sequence
  Oligonucleotide
ccctctggcggaggaggctcctgc

SEQ ID NO: 22 - DNA, 24 bp, Artificial Sequence
  Oligonucleotide
gcaggagcctcctccgccagaggg

SEQ ID NO: 23 - DNA, 24 bp, Artificial Sequence
  Oligonucleotide
ccctctgtcgaaggaggctcctgc

SEQ ID NO: 24 - DNA, 24 bp, Artificial Sequence
  Oligonucleotide
gcaggagcctccttcgacagaggg

SEQ ID NO: 25 - DNA, 24 bp, Artificial Sequence
  Oligonucleotide
ccctctggcgaaggaggctcctgc

SEQ ID NO: 26 - DNA, 24 bp, Artificial Sequence
  Oligonucleotide
gcaggagcctccttcgccagaggg

SEQ ID NO: 27 - DNA, 24 bp, Artificial Sequence
  Oligonucleotide
ccctctgccgaaggaggctcctgc

SEQ ID NO: 28 - DNA, 24 bp, Artificial Sequence
  Oligonucleotide
gcaggagcctccttcggcagaggg

SEQ ID NO: 29 - DNA, 24 bp, Artificial Sequence
  Oligonucleotide sequence 8 with A for G at 13
actggatccgccagcgctgaaggc

SEQ ID NO: 30 - DNA, 24 bp, Artificial Sequence
  Oligonucleotide sequence 8 with C for G at 13
actggatccgcccgcgctgaaggc

SEQ ID NO: 31 - DNA, 24 bp, Artificial Sequence
  Oligonucleotide sequence 8 with T fo G at 13
actggatccgcctgcgctgaaggc

SEQ ID NO: 32 - DNA, 24 bp, Artificial Sequence
  Oligonnucleotide sequence 8 with dU for G at 13
actggatccgccugcgctgaaggc

SEQ ID NO: 33 - DNA, 24 bp, Artificial Sequence
  Oligonucleotide sequence 8 with Inosine for G at 13
actggatccgccngcgctgaaggc

SEQ ID NO: 34 - DNA, 24 bp, Artificial Sequence
  Oligonucleotide with 5-MeC at 9
ccctctgacggaggaggctcctgc

SEQ ID NO: 35 - DNA, 24 bp, Artificial Sequence
  Oligonucleotide with 5-MeC at 15
gcaggagcctcctccgtcagaggg

SEQ ID NO: 36 - DNA, 24 bp, Artificial Sequence
  Oligonucleotide with 5-MeC at 9
ccctctgacgcaggaggctcctgc

SEQ ID NO: 37 - DNA, 24 bp, Artificial Sequence
  Oligonucleotide with 5-MeC at 15
gcaggagcctcctgcgtcagaggg

SEQ ID NO: 38 - DNA, 24 bp, Artificial Sequence
  Oligonucleotide with 5-MeC at 9
ccctctgacgaaggaggctcctgc

SEQ ID NO: 39 - DNA, 24 bp, Artificial Sequence
  Oligonucleotide with 5-MeC at 15
gcaggagcctccttcgtcagaggg

SEQ ID NO: 40 - DNA, 24 bp, Artificial Sequence
  Oligonucleotide with 5-MeC at 9
ccctctgacgtaggaggctcctgc

SEQ ID NO: 41 - DNA, 24 bp, Artificial Sequence
  Oligonucleotide with 5-MeC at 15
gcaggagcctcctacgtcagaggg

SEQ ID NO: 42 - DNA, 24 bp, Artificial Sequence
  Oligonucleotide with 5-MeC at 9
ccctctgccggaggaggatcctgc

SEQ ID NO: 43 - DNA, 24 bp, Artificial Sequence
  Oligonucleotide with 5-MeC at 15
gcaggatcctcctccggcagaggg

SEQ ID NO: 44 - DNA, 24 bp, Artificial Sequence
  Oligonucleotide with 5-MeC at 9
ccctctgtcggaggaggctcctgc

SEQ ID NO: 45 - DNA, 24 bp, Artificial Sequence
  Oligonucleotide with 5-MeC at 15
gcaggagcctcctccgacagaggg

SEQ ID NO: 46 - DNA, 24 bp, Artificial Sequence
  Oligonucleotide with 5-MeC at 9
ccctctggcggaggaggctcctgc

SEQ ID NO: 47 - DNA, 24 bp, Artificial Sequence
  Oligonucleotide with 5-MeC at 15
gcaggagcctcctccgccagaggg

SEQ ID NO: 48 - DNA, 24 bp, Artificial Sequence
  Oligonucleotide with 5-MeC at 9
ccctctgtcgaaggaggctcctgc

SEQ ID NO: 49 - DNA, 24 bp, Artificial Sequence
  Oligonucleotide with 5-MeC at 15
gcaggagcctccttcgacagaggg

SEQ ID NO: 50 - DNA, 24 bp, Artificial Sequence
  Oligonucleotide with 5-MeC at 9
ccctctggcgaaggaggctcctgc

SEQ ID NO: 51 - DNA, 24 bp, Artificial Sequence
  Oligonucleotide with 5-MeC at 15
gcaggagcctccttcgccagaggg

SEQ ID NO: 52 - DNA, 24 bp, Artificial Sequence
  Oligonucleotide with 5-MeC at 9
ccctctgccgaaggaggctcctgc

SEQ ID NO: 53 - DNA, 24 bp, Artificial Sequence
  Oligonucleotide with 5-MeC at 15
gcaggagcctccttcggcagaggg

SEQ ID NO: 54 - protein, 13 aa, Artificial Sequence
  StrepII Tag
MASWSHPQFEKGA

SEQ ID NO: 55 - protein, 10 aa, Artificial Sequence
  StrepII Tag
SAWSHPQFEK

SEQ ID NO: 56 - DNA, 23 bp, Artificial Sequence
  MseI-compatible adaptor
agcaactgtgctatccgagggat

SEQ ID NO: 57 - DNA, 12 bp, Artificial Sequence
  MseI-compatible adaptor (bottom)
taatccctcgga

SEQ ID NO: 58 - DNA, 20 bp, Artificial Sequence
  C1F3 forward primer
tggggtgttttttttgtatt

SEQ ID NO: 59 - DNA, 18 bp, Artificial Sequence
  C1R1 reverse primer
aaaatcccaccctaaacc

SEQ ID NO: 60 - DNA, 20 bp, Artificial Sequence
  C2F3 forward primer
tgggttttgtataggttaaa

SEQ ID NO: 61 - DNA, 20 bp, Artificial Sequence
  C2R1 reverse primer
aacaaccaaaaaattttcac

SEQ ID NO: 62 - DNA, 55 bp, Homo sapiens
atgccagcgggccgaagagtcgggacgggagccggagccgggagagcgaaaggag

SEQ ID NO: 63 - DNA, 30 bp, Homo sapiens
ctcctttcgctctcccggctccggctcccg

SEQ ID NO: 64 - DNA, 29 bp, Homo sapiens
gggagccggagccgggagagcgaaaggag

SEQ ID NO: 65 - DNA, 30 bp, Homo sapiens
  misc_feature (9)..(9): n is A, C, T or dU
ctcctttcnctctcccngctccngctcccg

SEQ ID NO: 66 - DNA, 30 bp, Homo sapiens
  misc_feature (9)..(9): n is A, C, T or dU
ctcctttcnctctcccngctccggctcccg

SEQ ID NO: 67 - DNA, 30 bp, Homo sapiens
  misc_feature (9)..(9): n is A, C, T or dU
ctcctttcnctctcccggctccngctcccg

SEQ ID NO: 68 - DNA, 30 bp, Homo sapiens
  misc_feature (17)..(17): n is A, C, T or dU
ctcctttcgctctcccngctccngctcccg